(19) United States
(12) Patent Application Publication
(10) Pub. No.: US 2018/0325719 A1
(43) Pub. Date: Nov. 15, 2018

(54) Urine Collection Bag with Sensor

(71) Applicant: Johnnie J. Hayes, Triangle, VA (US)

(72) Inventor: Johnnie J. Hayes, Triangle, VA (US)

(73) Assignee: Johnnie J. Hayes, Triangle, VA (US)

(21) Appl. No.: 15/731,240

(22) Filed: May 9, 2017

(51) Int. Cl. A61F 5/44

(52) U.S. Cl. CPC A61F 5/4404; A61M 2205/18

(57) ABSTRACT

The Urine Collection Bag With Sensor includes a connection tube from the to the urine bag to the catheter. The sensor is attached to the neck of the hooks that goes on the bed rail. The urine bag is made with a 3^{rd} hole with a tube in it allowing the sensor two wires to enter. These two wires are the wires that meet the urine allowing the sensor to detect the urine to sound off.

### BACKGROUND OF THE INVENTION

### (1) Field of the Invention

The present invention relates to a sensor for a urine bag, particularly to the sensor connected to the urine bag that sounds off alerting staff members that the bag is full. Even through that there are urine bags out there that have the reflux that prevent urine from back flowing the patent stills must go to the bathroom and when this happens the urine will sit in the line of the patients and eventually the patient will soil him or herself cause bed sores, UTI's and other problems. With this invention, it will prevent urine bags do to urine bags overfilling.

### (2) Description of the Prior Art

A general urine bag 1 is shown in FIG. 1 and generally includes a connection part 3 connected to one side of the urine bag 1 and a hose 3 is connected to the connection part 1. The hose is connected to the catheter which is connected to the user's body to guide the user's urine to the urine bag 1 via the hose 2. The urine can be collected or disposed when the urine bag 1 is full. However, there is no sensor connected to the urine bag 1 to alert staff that the is full to prevent the urine in the urine bag 1 from flowing back from the line to the user causing other problems. This is where 4 the sensor is connected to 8 the stem of the FIG. 1 having the two wires 5 inserted in 6 the other opening into the bag. Pressing 7 turning the off and on switch to on.

The present invention intends to provide a sensor on the urine bag by sounding off alerting staff members that the bag is full. With this sensor, it will help prevent the urine staying in the tube once the urine bag is full and back flowing and eventually having the patient soil him or herself or causing the patient to have a UTI.

### SUMMARY OF THE INVENTION

The present invention relates to a sensor that is attach to a urine bag and the sensor wire is inserting into the urine bag. When the urine fill up to maximum the sensor sounds off alerting staff members that it is full. This will allow the urine bag not to fill up all the way and have urine to sit in the line or sole the patient.

The primary object of the present invention is to provide a device on the urine bag that can inform staff member that the bag is full. Which would help prevent the urine bag from filling up with urine. This is to help prevent patients from UTI's and other infections and illnesses.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 shows a normal urine bag;

FIG. 2 is a perspective view to show the urine collection bag with sensor present invention; shows the connection from the sensor to the urine bag it also shows the urine bag at it maximum and the alarm sounding off.

FIG. 3 is the sensor by itself;

FIG. 4 is a view to show the wires and the connection tube are to be connected to the connection of the urine bag.

FIG. 5 shows the connection from the sensor to the urine bag

FIG. 6 shows tube coming out of the corner of the urine bag

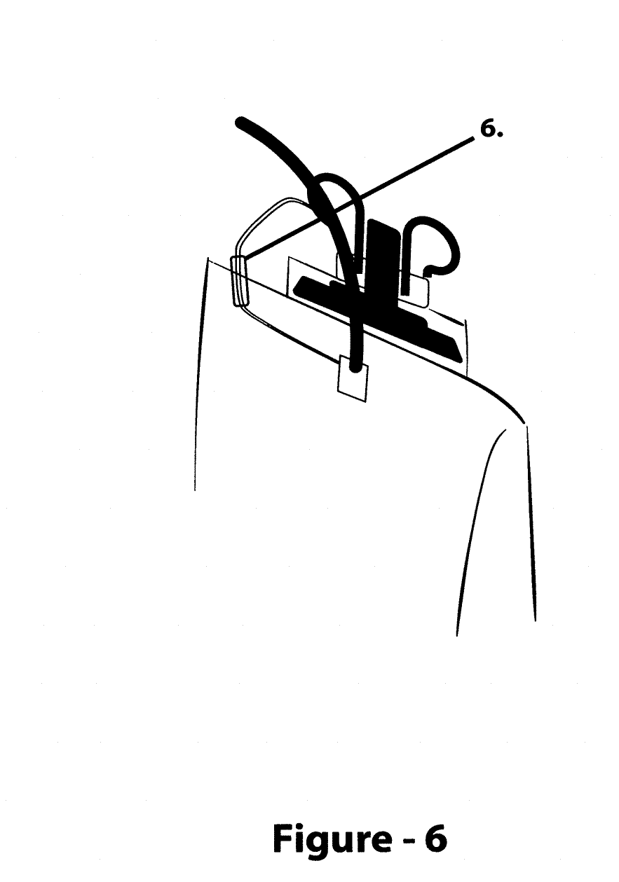

### DETAILED DESCRIPTION OF THE URINE COLLECTION BAG WITH SENSOR

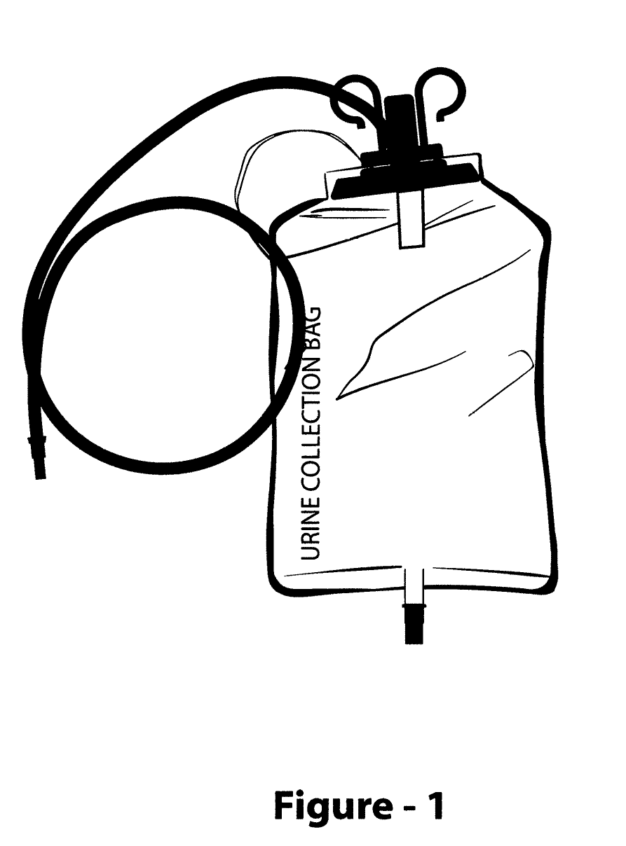

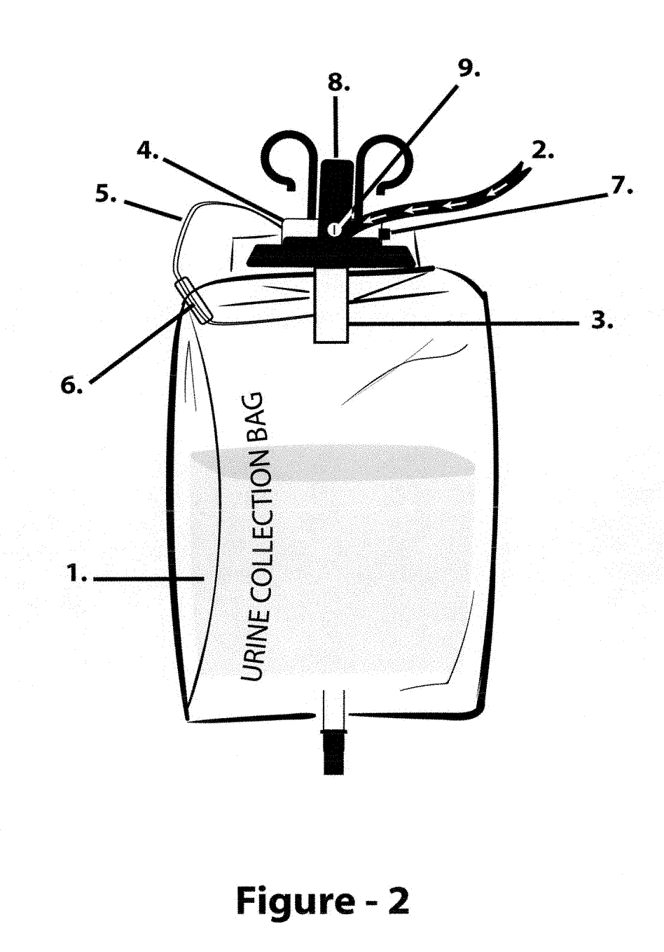

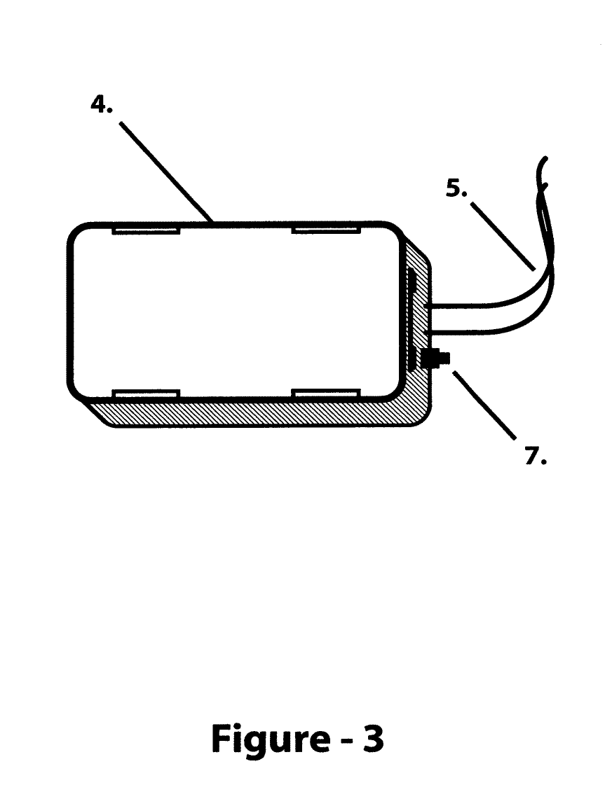

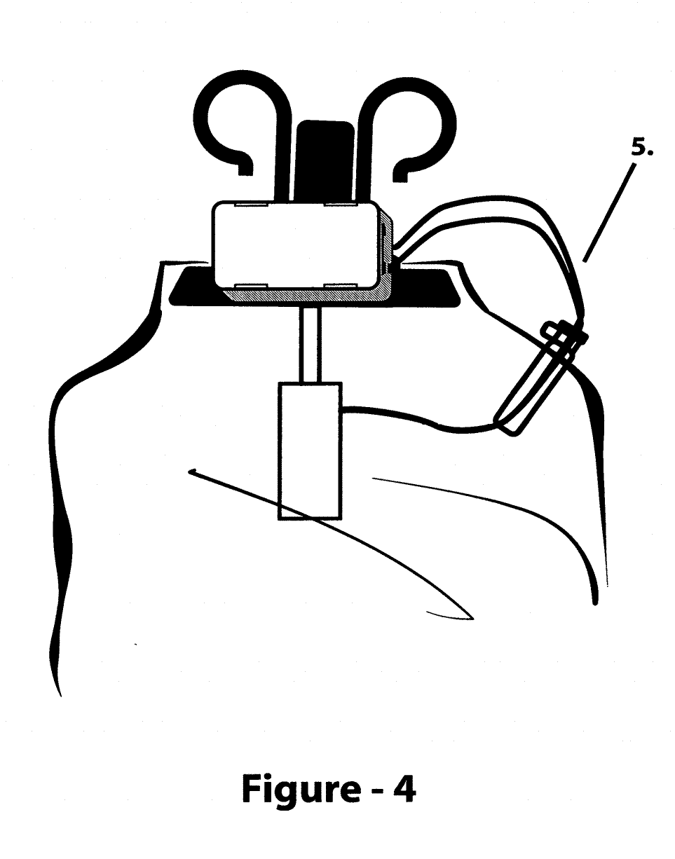

Referring to FIGS. 2 to 4, 1,2,3,4,5,6,7,8,9 shows a complete operational of the urine bag with sensor 5 sensor wire, 4 the sensor box, off and on switch 5 sensor wire that goes in into 6 the urine bag.

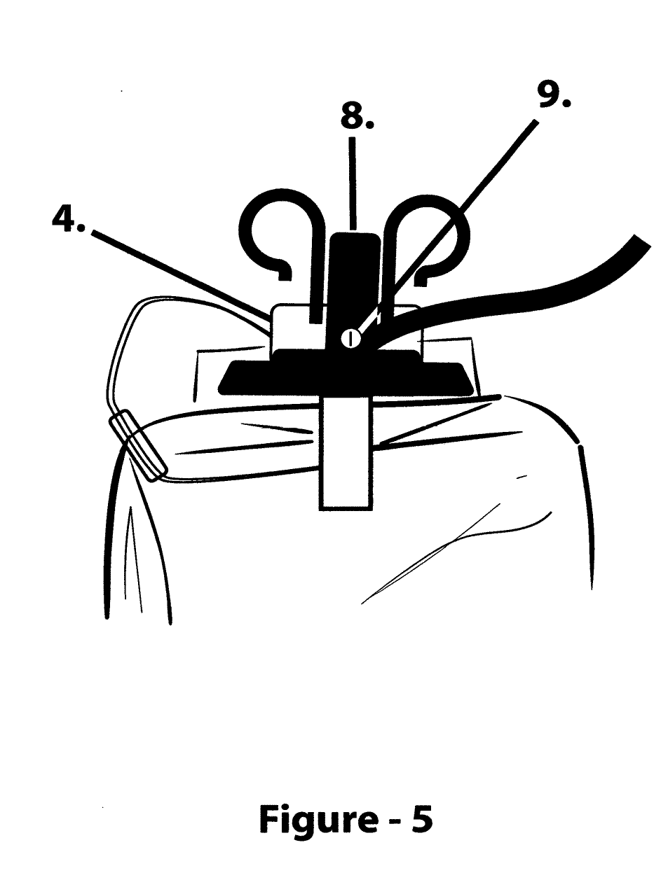

As shown in FIG. 5, the urine bag includes a connection part 9 to connect sensor to part 4 6 shows the tube coming out of the urine where the sensor wire is inserted.

1. A urine collection bag having a sensor attach to the urine bag with two wires that are inserted into the urine bag that sounds off when it comes in contact with the urine.

2. A urine collection bag with a third opening with a tube coming out where the two wires from the sensor gets inserted
